(19) United States
(12) Patent Application Publication
(10) Pub. No.: US 2025/0204950 A1
(43) Pub. Date: Jun. 26, 2025

(54) Percutaneous Renal Puncture System Improved Method, System and Apparatus

(71) Applicant: Jason B. Wynberg, Passaic, NJ (US)

(72) Inventor: Jason B. Wynberg, Passaic, NJ (US)

(21) Appl. No.: 19/002,206

(22) Filed: Dec. 26, 2024

Related U.S. Application Data
(60) Provisional application No. 63/614,892, filed on Dec. 26, 2023.

(51) Int. Cl. A61B 17/34; A61B 1/307; A61B 17/00; A61B 90/00

(52) U.S. Cl. CPC A61B 17/3415; A61B 1/307; A61B 17/00234; A61B 90/08; A61B 2017/00238; A61B 2017/00296; A61B 2017/00477; A61B 2017/00526; A61B 2017/00836; A61B 2017/0084; A61B 2017/00853; A61B 2017/00871; A61B 2017/00902; A61B 2017/00929; A61B 2017/00942; A61B 2017/00946; A61B 2090/0811

(57) ABSTRACT

This application relates to a retrograde percutaneous puncture system. The field of retrograde nephrostomy creation has an expanding use set and the current art has deficiencies. The present described system incorporates innovations such as a single-lumen exchange catheter system that simplifies exchange of a puncture wire for a larger diameter endourology working wire, various systems, methods, and several solutions to achieve direct force translation of an advancing puncture wire through to a distal puncture wire tip such as an apparatus that permits a sheathless puncture wire to be positioned in a ureteroscope working channel and elegant length preparation of a microcatheter sheath wire tip protector under sterile conditions prior to a retrograde nephrostomy puncture procedure to enhance stability of the puncture apparatus and force translation. Finally, the application describes adding functionality to a single lumen, multi-lumen or coaxial catheter loaded over the emergent puncture wire at the flank.

### BACKGROUND OF THE INVENTION

### Field of Invention

This application relates to a retrograde percutaneous renal puncture system. Various aspects of this field are known in the art. For example, systems to upsize wire diameters during a procedure are complex which can lead to errors. Additionally, the art describes a system for retrograde renal puncture that preserves surgical flexibility but does not consider force translation requirements of a wire advancement system. Several elegant solutions to this problem will be presented. Finally, a system to simplify and unify a renal nephrostomy puncture and a subsequent endourology procedure is lacking and is presented herein.

### Brief Summary of The Invention

The present described systems and apparatus innovations improve the function and performance, and add procedural enhancements to this field of endourology.

This patent describes a translucent or transparent single lumen catheter to facilitate replacement of an emergent narrower puncture wire with a larger diameter endourology puncture wire.

The patent addresses deficiencies in the prior art relative to global function of a retrograde renal puncture system in a surgical setting. Two general solutions to this problem are presented herein. First, a dual-function ureteroscope working channel that both guides wire deployment and serves as a puncture wire tip protector wherein a puncture wire is positioned in a ureteroscope without a separate tip protector. The puncture wire is secured to the ureteroscope directly. This system describes various methods for atraumatically positioning a puncture wire in a ureteroscope working channel and ensuring precise positioning of the puncture wire in relation to the ureteroscope. As well, design solutions include a flexible ureteroscope whose working channel is lined with puncture resistant materials such as a steel hypodermic tube. Second, preparation of a microcatheter sheath puncture wire tip protector prior to a retrograde nephrostomy puncture will elegantly convert multifunctionality of a microcatheter sheath to a stable and direct force translator of an advancing puncture wire to enhance performance and stability of the puncture apparatus during a retrograde nephrostomy puncture procedure.

The patent describes various enhancements to a single lumen, multi-lumen, or coaxial catheter loaded over an emergent puncture wire such as converting a puncture wire to a larger diameter endourology wire or catheter bridge, or urinary drainage or irrigation capabilities.

The patent also describes various kit configurations to support the above procedures, apparatuses, and systems.

### BRIEF DESCRIPTION OF THE FIGURES

FIG. 1A: An exchange catheter is loaded over the longer puncture wire segment that is emergent out of the patient, shown here at the urethral end.

FIG. 1B: After removal of the puncture wire, a second wire is advanced toward the tapered end of the exchange catheter until a length of second wire is seen through the wall of the translucent or transparent exchange catheter to be outside the patient. The exchange catheter is then removed in the same direction as the second wire was advanced, leaving the second wire emergent from the patient at the flank and urethra.

FIG. 2A: At the proximal end of the ureteroscope working channel, a releasable locking mechanism releasably secures the puncture wire to the ureteroscope and provides a watertight seal. Other functions of the releasable locking mechanism may include irrigation and an additional working port for laser introduction. The puncture wire may not easily pass through an angled segment of the working channel, such as the angle between the working channel in the ureteroscope handle and the offset proximal opening.

FIG. 2B: A sleeve-like accessory is shown here covering the distal sharp end of the puncture wire to prevent trauma during loading of the puncture wire through the ureteroscope working channel.

FIG. 2C: An alternate method to load a puncture wire into a ureteroscope working channel. Here, the puncture wire has been positioned in a ‘through and through’ position in a single lumen catheter which was positioned in a ureteroscopes working channel.

FIG. 2D: The sheath is drawn out of the distal end of the ureteroscope working channel, leaving the wire in the ureteroscope working channel. The wire position is finalized with the distal puncture tip just inside the end of the ureteroscope.

FIG. 2E: A ureteroscope is positioned in the renal calyx. A puncture wire is then advanced along a puncture-resistant ureteroscope working channel until the sharp point emerges from the ureteroscope tip.

FIG. 2F: A ureteroscope with puncture resistant working channel has greater resistance to active deflection and thus a wider curve radius at the ureteroscope tip. This curve permits advancement of a puncture wire around the flexed distal ureteroscope tip without the sharp point puncturing the working channel.

FIG. 3A: Excess microcatheter length relative to a ureteroscope working channel may result in unwanted movement of the releasable wire securing device during puncture wire advancement.

FIG. 3B: Trimming the excess microcatheter length positions the pin vise lock base against the ureteroscope apparatus while maintaining an ideal short 1-7 mm length of microcatheter to emerge from the ureteroscope tip. This results in direct force translation of puncture wire through the pinvise and ureteroscope working channel to the distal puncture wire tip during puncture wire advancement.

FIG. 4A: Exchange catheter with distal drainage holes and curl to secure catheter in renal pelvis.

FIG. 4B: Nephrostomy balloon loaded over a catheter (shown here) which has been loaded over the emergent puncture wire (not shown here).

### DETAILED DESCRIPTION

The invention relates to improvements to a renal percutaneous puncture system.

The invention includes a catheter with a minimum length to be through the urinary collecting system and emergent both urethrally and through the flank, whose distal catheter tip is tapered to have close to zero or minimal clearance to the puncture wire to permit advancement of the catheter over a puncture while passing through fascia and renal capsule. The inner diameter of the catheter along its length other than the tapered tip will be larger than the outer diameter of a standard endourology working wire e.g. 0.038 inches or 0.035 inches. The catheter is designed to simplify and reduce a myriad of potential errors and steps associated with exchange of a first puncture wire for a second larger diameter endourology working wire during a procedure. This exchange catheter may be constructed of a transparent or translucent material to allow a surgeon to confirm the position of a larger endourology wire inside the lumen of the catheter prior to removing the catheter.

The invention addresses deficiencies in prior art systems which prioritize surgical optionality with regard to a puncture wire tip protector that can be used to support a wire at the expense of stabilization of an advancing puncture wire. Two inventions are presented here to solve this problem.

First, positioning of a naked or sheathless puncture wire inside of a ureteroscope working channel. By securing a puncture wire directly to a ureteroscope, advancement of the puncture wire will result in preservation of force translation to the advancing puncture wire tip as it passes through tissue. This system specifically teaches positioning and securing of a puncture wire tip specifically in the pre-procedure setting 0-4 mm inside the distal tip of a ureteroscope working channel with a releasable locking mechanism that secures the puncture wire to the ureteroscope as well as a surgical method. It describes methods for atraumatically positioning a puncture wire in a ureteroscope working channel in a sterile pre-procedure environment utilizing an introduction cap on one end of the wire which is removed after the wire is introduced into the ureteroscope, a translucent or transparent single lumen catheter that enables through and through positioning of the wire through the ureteroscope working channel with visualization and precise positioning of the puncture wire under direct vision through the catheter wall, or simply narrowing the non-puncture end of the puncture wire to create a flexible tip that can be loaded first into a ureteroscope working channel where the flexible tip navigates through the ureteroscope working channel without damaging the channel. Additionally, it contemplates a procedure wherein a puncture wire is releasably secured directly to a ureteroscope, released in relation to the ureteroscope after the ureteroscope is positioned in the kidney, and then the wire is advanced from the ureteroscope through a portion of the kidney and out the flank to enable an endourology procedure. Finally, it contemplates a ureteroscope design with a puncture-resistant working channel which may incorporate a puncture resistant tube (e.g. stainless steel) inserted into the ureteroscope working channel during ureteroscope manufacturing assembly to enhance durability of the working channel thus allowing a puncture wire to be inserted directly into the working channel of the ureteroscope. These features enable the positioning of a ‘naked’ puncture wire into a flexible ureteroscope without damaging the working channel or compromising procedural safety.

Second, current art presents a puncture wire tip protector microcatheter with an attached wire securing mechanism which fixes a puncture wire tip in relation to the distal end of the wire tip protector microcatheter. This device is mobile in relation to a ureteroscope whose working channel critically translates the force of puncture wire advancement from outside the proximal end of the working channel to the puncture wire tip emergent from the distal end of the ureteroscope. This mobility affords a physician some advantages, however, there is a loss of critical preservation of force translation of the advancement of the puncture wire through the ureteroscope working channel. This is owing the too-long microcatheter length in relation to the ureteroscope working channel. This excess length of the microcatheter with luminal puncture wire results in buckling of this segment as the puncture wire is advanced and also creates unwanted motion of the pin vise lock and motion of the pin vise lock during advancement of the puncture wire. The current invention elegantly corrects this loss-of-force problem by customizing a puncture wire tip protector microcatheter length to a specific ureteroscope working channel in a pre-procedure sterile environment that will result in the pin vise lock resting in a preferred position against the ureteroscope irrigation port resulting in direct force translation to the distal puncture wire tip during puncture wire advancement. This series of steps involves cutting excess microcatheter length to a specific ureteroscope/irrigation port configuration being used in a specific procedure. This can be done by direct measurement of the ureteroscope working channel and the microc catheter and cutting excess microcatheter from its distal tip or else inserting a microcatheter into a working channel of a sterile ureteroscope working channel with an attached irrigation port at the top of the ureteroscope working channel until the pin-vise hub is resting on the ureteroscope irrigation port. The length of microcatheter that is emergent from the distal end of the ureteroscope represents excess length of microcatheter. At this time, if a wire is in the microcatheter, the pin vise lock is opened and a length of puncture wire is drawn out of the catheter leaving the segment of microcatheter that is outside the distal end of the ureteroscope without a wire in its inner lumen. Confirming that the pin vise lock is resting on the ureteroscope irrigation port, the microcatheter is cut sharply, leaving between 0-8 mm of microcatheter outside of the ureteroscope and more likely 3-4 mm outside.

Separately and finally, the art describes a coaxial exchange catheter that is loaded over an emergent puncture wire at the flank for the purpose of exchanging a narrower puncture wire for a larger endourology wire in a controlled fashion, in the setting of nephrostomy creation. In this invention, a plurality of catheters with various functions are contemplated, each of which would be loaded directly over an emergent puncture wire. These catheters may be single lumen, multi-lumen or coaxial catheter and may terminate in the renal pelvis, ureter, or bladder. These catheters may provide specific functions such as (i) instillation of irrigation fluids (e.g. to cool the kidney during high temperature laser or other therapeutic treatments), (ii) instillation of diagnostic or therapeutic medications e.g. chemo/immunotherapy, ICG dye or contrast for imaging) or gaseous media, (iii) to measure pressure or temperature in the urinary collecting system, (iv) facilitate stenting of a narrowed ureter or removal or treatment of stones obstructing the ureter, (v) serve as a conduit to luminally introduce small video capability e.g. endoscope or small instrumentation, (vi) drainage of the urinary collecting system e.g. nephrostomy or nephrovesical tube placement, or (vii) to load a nephrostomy balloon directly over top of the catheter. In this final scenario, over the emergent ‘through and through’ puncture wire a catheter is loaded. This catheter will increase the diameter around the puncture wire that interfaces with the patients tissues e.g. collecting system, mucosa, renal tissue, thus reducing risk of renal injury during endourologic procedure that would otherwise be caused by a thin puncture wire passing across patient tissues, and increases kink resistance of the puncture wire. By increasing the diameter and thus reducing the risk of injury to renal tissues, an endourologic procedure can be performed safely over this catheter e.g. load a nephrostomy balloon over this catheter e.g. for PCNL. Also, by eliminating the need for a second endourology wire the endourology procedure is streamlined. In this embodiment, the puncture wire can either be maintained inside the catheter loaded over it or the puncture wire can be removed prior to or during the endourological procedure.

Kit configurations described in the invention provide a comprehensive solution for the procedure. They may include a nephrostomy puncture wire, a single lumen exchange catheter, sleeve-like device or translucent or transparent catheter to atraumatically introduce a puncture wire into a ureteroscope, and accessories like a Touhy Borst connector for securing the wire to the ureteroscope possibly with additional side port functions. Additionally, the kits may incorporate a specialized flexible ureteroscope with a puncture-resistant working channel or any number of catheters with different functions that may be loaded directly over an emergent puncture wire at the flank—e.g. nephrostomy catheter, nephrovesical catheter, sheath for luminal introduction of an endoscope, etc.

### Detailed Description of Figures

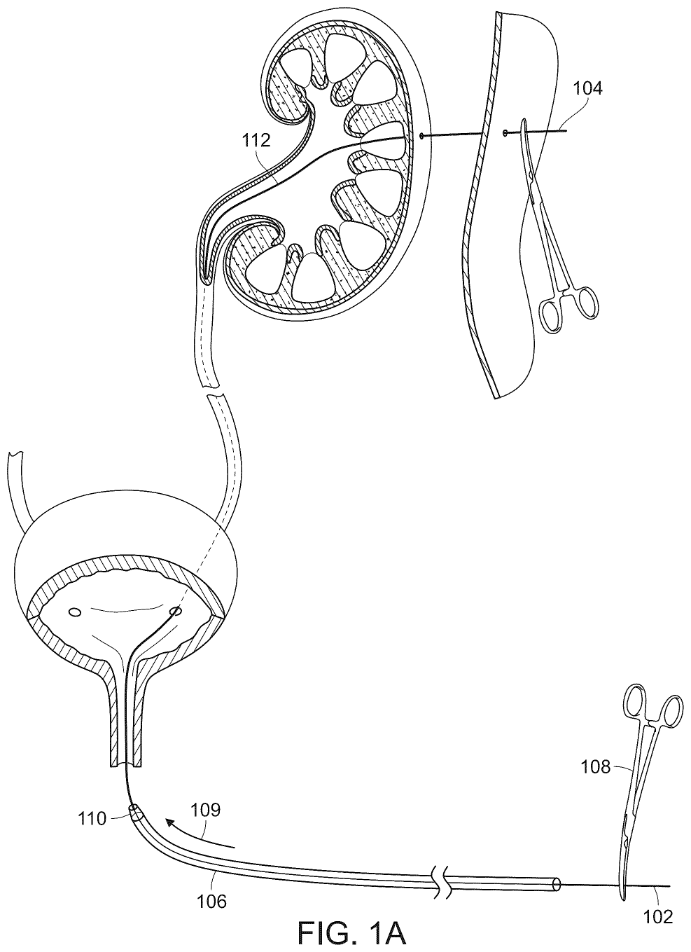

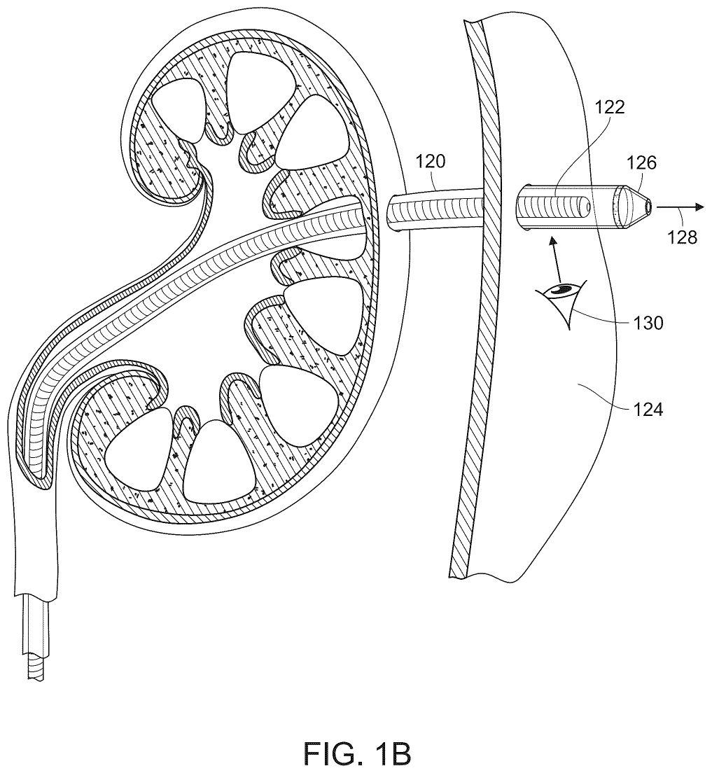

FIG. 1A: After a nephrostomy puncture is created a first puncture wire 112 is emergent both at the urethral 102 and flank end 104, for example after a ureteroscopy-assisted retrograde nephrostomy procedure. To effect a wire exchange in favor of a second larger diameter, kink resistant endourology wire in a controlled and safe fashion, an exchange catheter 106 is loaded over the longer segment of puncture wire that is emergent out of the patient whether the longer segment is emergent at the flank or urethra. The emergent puncture wire is then controlled 108 behind the loaded exchange catheter. In the setting of a ureteroscopy-assisted retrograde nephrostomy procedure the ureteroscope and any other puncture apparatus are removed from the patient prior to loading an exchange catheter over the puncture wire which is in a ‘through-and-through’ position. If the exchange catheter has a narrow tip 110, the narrow tip end is loaded first over the emergent first puncture wire so that when the catheter is advanced, this tapered tip will dilate the tissue it travels through atraumatically and with reduced resistance. The first puncture wire 112 will be of sufficient length to be (i) emergent and controlled on one end, shown here at the flank 104, (ii) position the full length of the exchange catheter 106 over the emergent wire 102, and (iii) emergent behind the exchange catheter to enable control 108 all before the catheter is advanced 109 into the patient, thus the puncture wire will have a minimum length of 130 cm and more likely be between 140 and 220 cm. The catheter is advanced until it is emergent from both the urethral and flank end (not imaged here). The puncture wire is then removed, leaving the exchange catheter in a ‘through-and-through’ position, emergent from urethra and flank. The second larger diameter endourology working wire is then introduced into a non-tapered end of the exchange catheter and advanced toward the other end of the exchange catheter.

FIG. 1B: Imaged here is the exchange catheter loaded tapered tip first into the urethra and advanced retrograde until it emerged out the flank. The exchange catheter could also have been loaded in the other direction to emerge at the urethra, not shown here. In this scenario shown here of retrograde loading of the exchange catheter, the exchange catheter was advanced out of the flank and the first puncture wire was removed. Then a second larger diameter endourology wire was advanced into the urethral non-tapered end of the exchange catheter and advanced until it entered a segment of exchange catheter that is outside the flank. If the exchange catheter 120 is translucent or transparent, the surgeon can visually confirm through the emergent segment of exchange catheter 130 that the luminal second wire 122 has advanced out of the patient's flank 124, thus achieving an intended ‘through and through’ position of a second wire. The second wire in this scenario does not necessarily pass through the narrowed catheter tip 126 as the catheter tip may be narrower than the OD of the second wire. Other ways to confirm the second wire 122 has emerged from the flank 124 or urethra (depending on direction of catheter advancement over puncture wire) are by palpation through the catheter or by fluoroscopy, not shown here. At this time, the tapered tip of the catheter 126 may be cut off with care not to damage the luminal second wire, thus allowing advancement of the second wire through the emergent catheter to aid in control of the wire. An alternate approach that does not involve cutting off the tapered tip of the catheter is to remove the exchange catheter 120 from the patient in the same direction 128 as the second wire was advanced (shown here in the retrograde fashion) while confirming e.g. visually through the translucent exchange catheter wall 130 that a length of the second wire 122 is outside the patients flank 124. In this scenario, the catheter's tapered tip is drawn away 128 from the wire 122 until the catheter has been completely removed from the patient, leaving the second wire ‘through-and-through’ the patient.

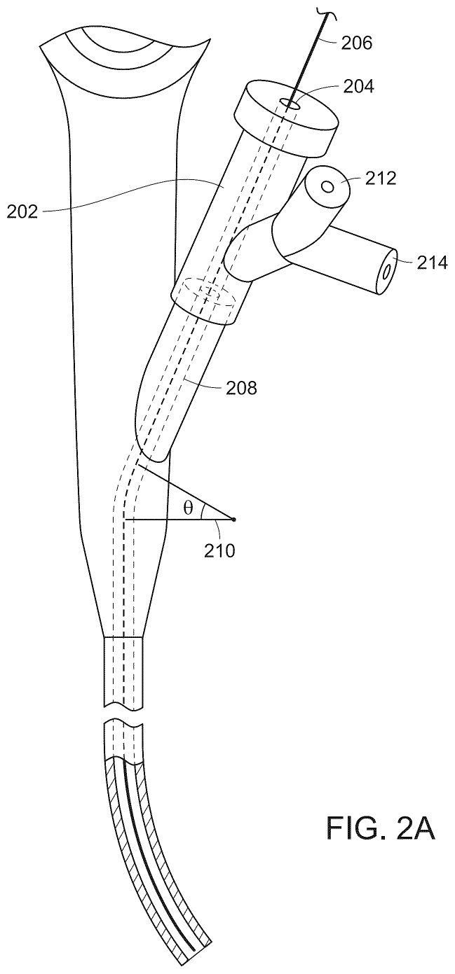

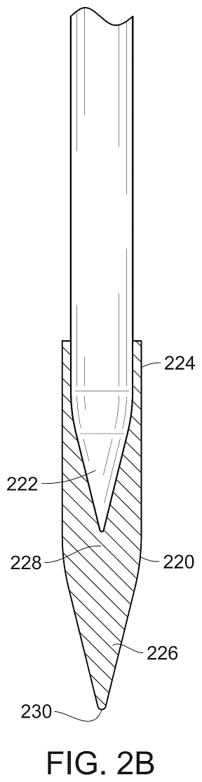

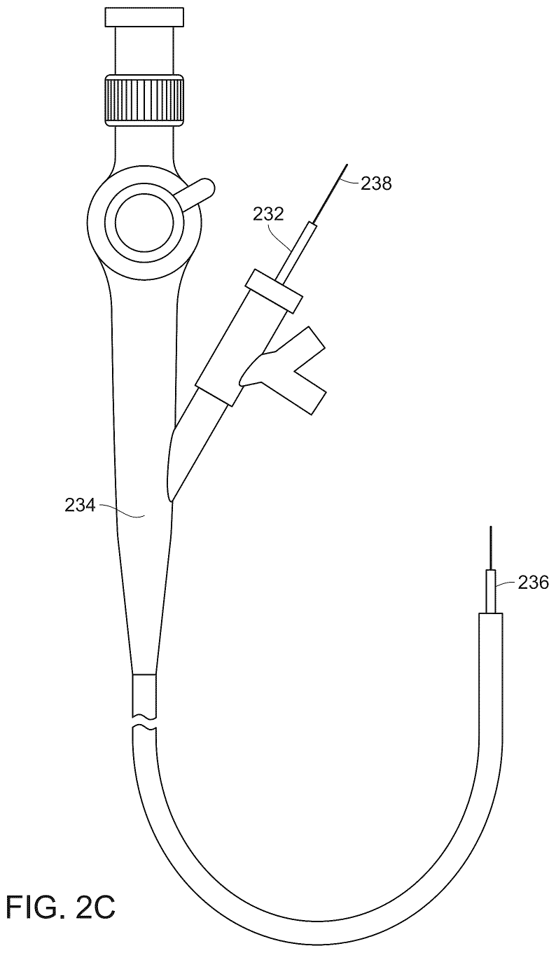

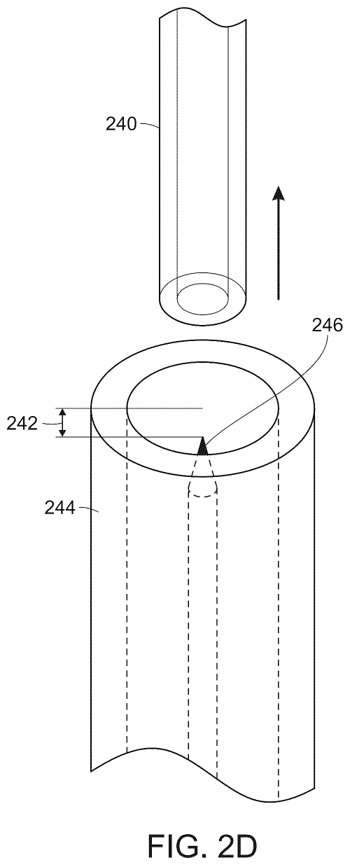

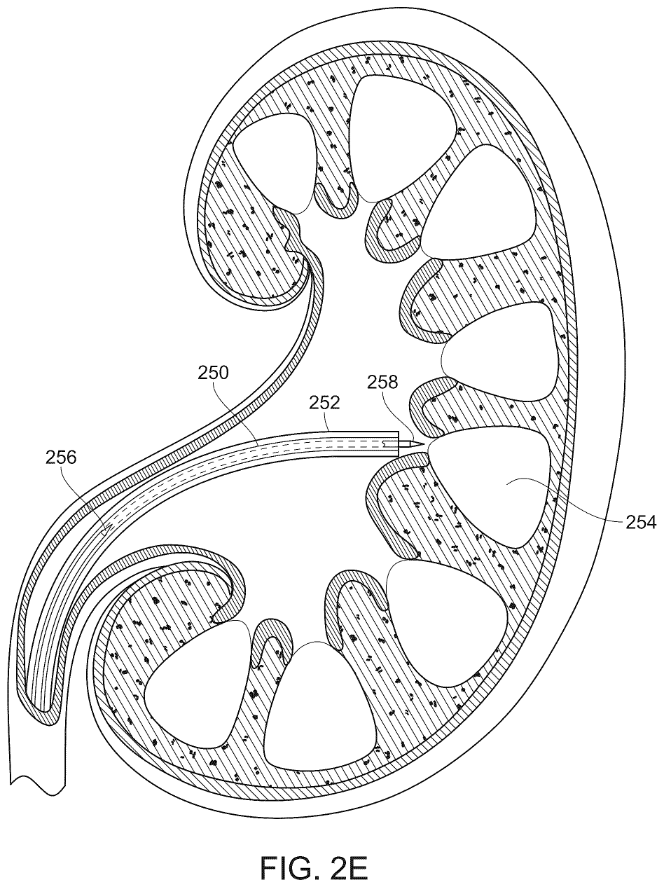

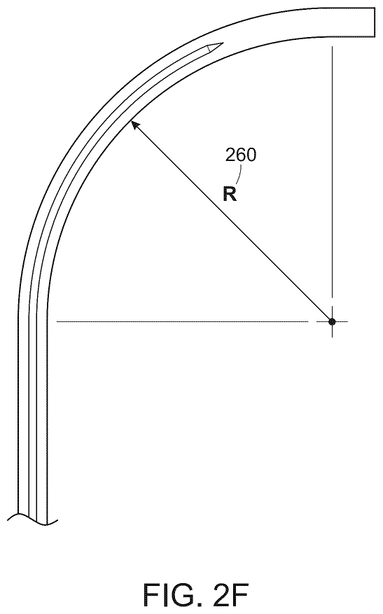

FIG. 2A: At the proximal end of the ureteroscope working channel, typically near the handle of the flexible ureteroscope, a releasable locking mechanism is provided or pre-attached to the top entry of the ureteroscope working channel 202 (e.g., Touhy-Borst cap) that releasably secures the sheathless puncture wire to the ureteroscope and provides a watertight seal 204. A releasable locking mechanism 202 may have additional accesses to permit holmium laser introduction 212 and irrigation 214. During equipment preparation under sterile conditions just prior to surgery or during manufacturing, introduction of a sheathless puncture wire 206 into the flexible ureteroscope working channel 208 can result in scraping and damage to the lining of the ureteroscope working channel whether the sharp point or blunt end of the wire is loaded first. Additionally, the wire may not easily pass through an angled segment of the working channel, such as the angle 210 between the working channel in the ureteroscope handle and the offset proximal opening of the working channel.

FIG. 2B: A sleeve-like accessory 220 may be fitted over the proximal blunt end or alternatively the sharp distalmost end of the puncture wire (shown here, 222) during loading of the wire into the ureteroscope. The sleeve-like accessory has two properties along its length: a ‘wire end’ and a ‘beyond wire end’ property, which differ from one another. The ‘wire end’ of the accessory 224 has a tubular configuration to fit over the puncture wire end and thereby releasably secures the accessory to the end of the wire (e.g. snug fit). The ‘non-wire end’ 226 property of the accessory is to (i) resist advancement of the wire 222 through the accessory 228, and (ii) have a flexible atraumatic tip 230 that will navigate through the working channel of the ureteroscope. This sleeve can be created via mold or other manufacturing methods and using any range of materials-silicone, PTFE, plastic, polymers, nylon, metal, etc. With the ‘wire end’ end of the sleeve 224 in place over the end of the wire 222, the ‘non-wire end’ of the sleeve 226 is loaded into the ureteroscope working channel and advanced by advancing the wire until the sleeve and wire emerge sufficiently from the opposite opening of the ureteroscope working channel. The sleeve apparatus is then removed leaving the puncture wire ‘through-and-through’ the ureteroscope. The puncture wire's final position in the ureteroscope working channel can then be set and the releasably securing mechanism 204 can be tightened around the wire 206. This process can be conducted during a manufacturing process prior to sterilization of the ureteroscope or in an operating room under sterile conditions prior to the nephrostomy creation procedure.

FIG. 2C: In this embodiment a transparent or translucent sheath 232 that is longer than the length of the ureteroscope working channel is loaded into the ureteroscope working channel until the sheath is emergent out both the proximal 232 and the distal end 236 of the ureteroscope 234. The puncture wire 238 is advanced into the loading sheath, likely while the ureteroscope is in a straight position (i.e. ureteroscope tip is not flexed) until the wire 238 is ‘through-and-through’ the single lumen catheter 232 and therefore ‘through-and-through’ the working channel of the ureteroscope 234. The catheter 232 is then removed leaving the puncture wire 238 in position ‘through-and-through’ the ureteroscope. The puncture wire's final position in the ureteroscope working channel can then be set and the releasably securing mechanism 204 can be tightened around the wire 206. This process can be conducted during a manufacturing process prior to sterilization of the ureteroscope or in an operating room under sterile conditions a short time before the nephrostomy creation procedure.

FIG. 2D: At the conclusion of a wire loading into a ureteroscope using a sheath 232 above, one precise method of completing a final step of positioning a puncture wire tip 246 precisely in the distal ureteroscope working channel 244 is as follows. After (i) an exchange catheter that was pre-positioned ‘through and through’ a ureteroscope working channel 232, and (ii) a puncture wire 238 is inserted into the sheath and emergent from both ends of the catheter and thus the ureteroscope working channel, (iii) a first length of the single lumen catheter is drawn out of the distal working channel until the releasable lock can contact the puncture wire directly 204. The translucent or transparent construction of this catheter 240 permits visual confirmation of positioning the puncture wire tip 246 1-2 mm 242 inside the end of the ureteroscope working channel 244 after which the wire position can be secured by closing the locking mechanism 204. The wire can be locked 204 either before or after the catheter is completely removed from overtop the distal puncture wire tip.

FIG. 2E: The working channel of the ureteroscope may benefit from puncture-resistant coatings or linings 250 to increase puncture resistance of the working channel and possibly reduce internal angles of the working channel. This can be accomplished by various means such as integrating into the manufacturing process materials like steel, titanium, thermoset plastics, or polymers. These coatings or linings are designed to minimize trauma and ensure smooth passage of instruments, especially in angled or deflected regions. Another method to enhance the puncture resistance of the ureteroscope working channel is to incorporate a pre-extruded or pre-built puncture-resistant tube into the ureteroscope working channel during the ureteroscope manufacturing process. This tube may be composed of materials such as Teflon, steel, or other durable or composite metals or polymers, which provide both lubricity and puncture resistance. Here the ureteroscope 252 is positioned facing a renal papilla 254 to initiate the retrograde nephrostomy puncture procedure. After positioning the ureteroscope, an unsheathed, sharp-point puncture wire is introduced into the puncture-resistant ureteroscope working channel and advanced 256 through the channel 250 until the sharp point emerges from the ureteroscope tip 258. The wire is then guided through the renal papilla and flank of the patient for precise nephrostomy tract creation. Puncture-resistant working channels ensure durability and integrity of the ureteroscope working channel during the wire advancement portion of the procedure.

FIG. 2F: A ureteroscope working channel with puncture resistant features may produce a wider bend radius 260 of the ureteroscope tip during active flexion compared to standard commercially available ureteroscopes. This wider radius may be due to the materials adding puncture resistance to the working channel lining and may fortuitously permit the successful advancement of a puncture wire around flexed segments of the ureteroscope without puncturing the working channel.

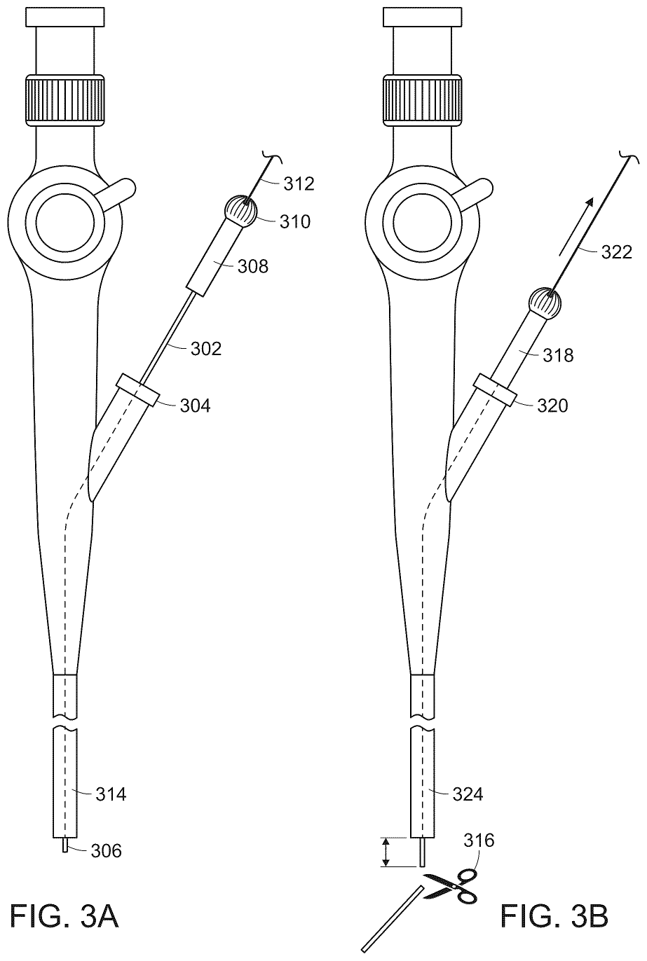

FIG. 3A: Flexible ureteroscopes are designed with varying working channel lengths. Additionally, various Luer irrigation port devices are used which are attached to the proximal end of the ureteroscope. The range of lengths of both a ureteroscope and Luer irrigation port together create a wide range of total working channel length of the ureteroscope/Luer device configuration. To accommodate all possible ureteroscope length configurations and to permit a range of surgical advantages and functions, a longer microcatheter (e.g. of Teflon construction) is selected for manufacturing. The result is that when used in most ureteroscope configurations the microcatheter is significantly longer than the combined effective ureteroscope working channel length (ureteroscope+Luer adaptor). This preserves certain surgical optionality for a surgeon (e.g. adding buttress and stability support of a microcatheter advanced over a puncture wire advancing through tissue) but misses a critical function of preservation of wire advancement force translation through the apparatus to the emergent distal puncture wire tip. The optimal emergent length of the microcatheter from the distal end of the ureteroscope is 1-5 mm. This favorable configuration permits direct vision of the wire advancing from the microcatheter (to confirm the sharp wire point will be advanced outside of the catheter only after it is beyond the ureteroscope) while maintaining the position of the ureteroscope tip in the renal calyx. A longer length of microcatheter emergent distally will risk pushing the microcatheter against the renal papilla, causing the ureteroscope tip to dislodge from the calyx in a direction opposite the puncture or else, if the microcatheter is advanced maximally into renal tissue, can cause renal trauma. To avoid this, a surgeon positions any excess microcatheter length 302 above the ureteroscope 304 to maintain 1-5 mm of microcatheter outside the distal end of the ureteroscope 306. This results in instability of the apparatus and loss of force translation of the wire, in addition to ergonomic complexity: The segment of excess microcatheter 302 and the puncture wire within this segment of catheter (not shown here) that are between the ureteroscope 304 and the puncture apparatus pin vise lock 308 is quite mobile and bendable. Opening of the pin vise cap 310 to advance the puncture wire 312 results in unwanted motion of the pin vise lock 308 in relation to the ureteroscope 304 as well as potential lateral herniation or column buckling of the excess segment of microcatheter 302 between the pin vise and the ureteroscope. The motion of the pin vise in relation to the ureteroscope is a distraction for a surgeon and challenge for the assistant, and the herniation of the microcatheter/wire reduces force translation of puncture wire advancement 312 force to the emergent sharp puncture wire out of the microcatheter segment 306 emergent from the distal end of the ureteroscope 314 making renal puncture less effective. As well, if the pin vise lock 308 slides downward toward the ureteroscope 304 during the puncture wire advancement process there will be an length of microcatheter advancing over the puncture wire possibly into the renal tissue. Again, these collective effects, though they offer specific benefits for a surgeon, can create distraction for the surgeon and impaired puncture performance due to force loss. Thus the excess length of microcatheter in relation to a specific ureteroscope configuration must be eliminated prior to a puncture procedure to prevent such complications and to optimize force translation performance of the puncture apparatus during the renal puncture surgery.

FIG. 3B: To solve these problems, the catheter is trimmed 316 to match the total working channel length, allowing the pin vise lock 318 to rest stably against the Luer apparatus 320 on the proximal entry to the working channel thus maximizing force translation of wire advancement and unit stability during puncture. Shown here is a scenario of trimming procedure conducted inside the ureteroscope working channel, though this trimming procedure does not require that the catheter be trimmed while inside the working channel. In this image, the pin vise/puncture apparatus is inserted in the ureteroscope until the pin vise 318 rests on the Luer apparatus on the proximal ureteroscope working channel opening 320. A length of puncture wire 322 is drawn back to remove the distal end of the puncture wire from the distally emergent microcatheter. The ‘empty’ microcatheter outside of the ureteroscope tip 324 is then trimmed 316 leaving an optimal, short length of catheter outside the distal end of the ureteroscope. The trimming process can also be conducted in the operating room or during manufacturing to tailor the catheter to a specific, stable ureteroscope configuration. Once the microcatheter has been trimmed 316, the distal puncture wire sharp point is repositioned inside the distal end of the microcatheter and the pin vise cap 310 is closed to secure the wire position in the catheter. The puncture wire, microcatheter, pin vise apparatus are now optimized for puncture wire advancement during the subsequent retrograde nephrostomy creation procedure.

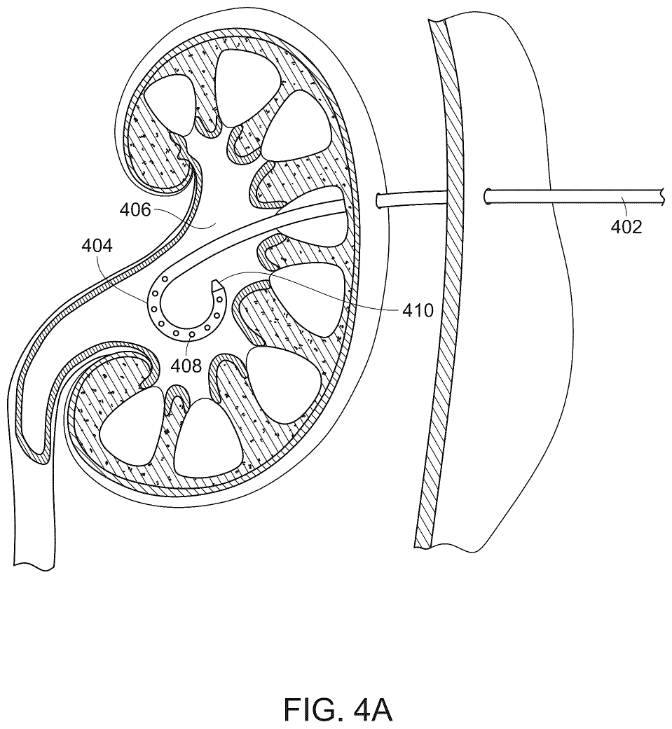

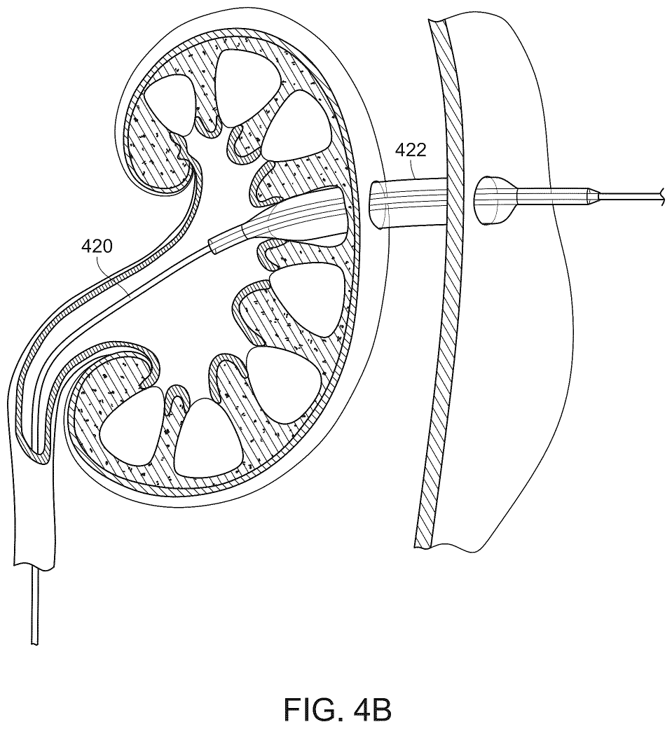

FIG. 4A: A single lumen, dual lumen or coaxial catheter of various functionality can be loaded directly over a ‘through-and-through’ puncture wire emergent at the flank (not shown here), where such catheters can be used for various purposes, e.g. irrigation/instillation or drainage purposes (shown here). This concept adds a bridge-like function to a segment of emergent puncture wire. Shown here is a positioned catheter that was loaded over an emergent puncture wire (not shown here), after removal of the puncture wire, leaving the enhanced function catheter in place. The catheter shown here 402 may have a curl 404 to secure the catheter in the renal pelvis 406 and may have drainage holes 408 along its distal segment and a tapered tip 410 to ease advancement through flank tissues. In another embodiment of a functional catheter loaded over an emergent puncture wire at the flank, here (FIG. 4B) a single lumen catheter 420 is loaded over the puncture wire (not shown here) to increase the overall diameter around the puncture wire to reduce risk of slicing renal tissue during the endourologic procedure (such as when there is tension on a wire against tissue) and to increase the kink resistance of the puncture wire. This catheter 420 has an inner diameter larger than the puncture wire diameter to permit loading over the puncture wire, and the catheter 420 may have an outer diameter that is between 0.024 and 0.050 inches but preferably between 0.034 and 0.042 inches. This catheter likely has tapered ends to smooth advancement over the puncture wire through the tissues. The puncture wire could be maintained in the lumen of the dilator or removed prior to the ongoing endourology procedure. As shown here, a nephrostomy balloon 422 is loaded over the catheter 420. By having advanced this catheter 402 over the emergent first puncture wire, an endourology procedure can be commenced without the need for a second endourology working wire. This represents a potential cost savings and reduces the number of procedural steps.

What is claimed is:

1. A method of retrograde nephrostomy creation and subsequent wire exchange:
a) Perform a renal puncture resulting in a first puncture wire emergent at patient's flank and urethra;
b) Advance a single lumen catheter over an emergent puncture wire until a portion of the catheter is emergent outside the urethra and flank;
c) Remove the first puncture wire from within the catheter leaving the catheter in position;
d) Insert a second wire luminally in the catheter until a portion of a second wire is outside the urethra and flank;
e) Remove the catheter leaving a second wire in position whose ends are outside both urethra and flank.

2. An apparatus of claim 1, wherein the catheter is translucent or transparent.

3. An apparatus of claim 1, wherein the catheter has a tapered tip to penetrate tissue when advanced over the first puncture wire.

4. A method of claim 1, wherein the renal puncture is performed using a ureteroscopy assisted retrograde nephrostomy creation technique.

5. A method of claim 4, wherein a ureteroscope is removed prior to loading a catheter over an emergent first puncture wire.

6. A method of claim 1, wherein an exchange catheter is loaded over the puncture wire outside the urethra and advanced in a direction to advance up the ureter and then through flank tissues and out the flank.

7. A method of claim 1 (e), wherein the exchange catheter is removed in the same direction as the second wire was advanced.

8. A method of claim 1 (d), wherein the second wire is of larger diameter than the first puncture wire.

9. A method of claim 1 (d), wherein prior to inserting a second catheter the tapered tip is cut off to leave the entire inner diameter of the catheter large enough for passage of the second wire.

10. A method of atraumatically positioning a puncture wire in a ureteroscope working channel prior to a nephrostomy creation procedure:
a) inserting a first portion of a single lumen catheter into a ureteroscope working channel;
b) advancing a puncture wire into a ureteroscope working channel where the leading edge of the puncture wire is inside the lumen of the catheter as the leading edge of the puncture wire travels the entire length of the working channel;
c) removal of the single lumen catheter leaving the puncture wire in the ureteroscope working channel.

11. The apparatus of claim 10, wherein the single lumen catheter is constructed of a clear or translucent property to permit the positioning of the distalmost sharp point of the puncture wire 1-2 mm inside the distal end of the ureteroscope working channel under direct vision by visualizing the puncture wire point through the single lumen catheter.

12. The method of claim 10, wherein after the wire tip is set in position 1-2 mm inside the distal end of the ureteroscope, the first segment of puncture wire emergent from the proximal end of the ureteroscope wire securing mechanism at the handle is marked with a marker to permit repositioning of the wire inside the ureteroscope tip by using the marked wire segment to indicate puncture tip position distally.

13. A method for creating a nephrostomy tract comprising the steps of:
a) providing a puncture wire having a tissue penetrating tip pre-positioned inside the ureteroscope working channel directly and releasably lockingly engaged to the ureteroscope where the sharpened distal tip of the puncture wire is positioned at or inside the working channel opening at the distal end of the ureteroscope and is releasably secured in this position in relation to the ureteroscope;
b) advancing the ureteroscope into the ureter and to the selected calyx;
c) releasing the puncture wire from its secured position in relation to the ureteroscope and advancing the puncture wire a first distance;
d) advancing the puncture wire through the papilla and through the flank of a patient.

14. The apparatus of claim 10, further comprising:
(a) Nephrostomy puncture wire;
(b) Single lumen catheter whose ID is 0.016-0.026 inches, whose OD is less than 0.039 inches; length 80-120 cm;
(c) Touhy Borst connector to permit watertight securing of a puncture wire directly to a ureteroscope.

15. The apparatus of claim 10, further comprising:
(a) Nephrostomy puncture wire;
(b) Single lumen catheter whose ID is 0.016-0.026 inches, whose OD is less than 0.039 inches; length 80-120 cm;
(c) Touhy Borst connector to permit watertight securing of a puncture wire to ureteroscope;
(d) Exchange catheter to permit exchange of puncture wire for larger diameter second wire-serial dilators vs. coaxial dilator.

16. A method of product preparation that customizes a microcatheter/pin vise apparatus to a specific ureteroscope configuration to optimize performance during a ureteroscopy-assisted retrograde nephrostomy puncture procedure:
a) Measure the length of the microcatheter from base of releasable wire securing mechanism to distal tip of microcatheter;
b) Measure the length of the ureteroscope working channel from the top of an irrigation seal to the tip of the ureteroscope working channel;
c) Trim the distal segment of microcatheter so that the final length of the microcatheter will be 0-7 mm longer than the ureteroscope working channel length;
d) Position a puncture wire point 0-5 mm proximal to the distalmost point of the cut microcatheter and secure the wire position by closing a releasable wire securing mechanism lock against the wire.

17. A method of claim 16, wherein the distal excess segment of microcatheter is trimmed by inserting the microcatheter into a ureteroscope working channel until the releasable wire securing mechanism that is secured to the microcatheter puncture wire tip protector rests directly against the irrigation port of the ureteroscope and then trimming excess microcatheter that is emergent from the distal end of the ureteroscope.

18. An apparatus of claim 16, wherein the microcatheter wire tip protector is composed of PTFE—polytetrafluoroethylene.

19. An apparatus of claim 16, wherein the releasable wire securing mechanism attached to a microcatheter is a pin vise lock.
